(19) United States
(12) Patent Application Publication
(10) Pub. No.: US 2023/0257402 A1
(43) Pub. Date: Aug. 17, 2023

(54) FUNCTIONALIZED FLUOROALKYL SILANE, AND SYNTHETIC METHOD THEREFOR AND APPLICATION THEREOF

(71) Applicant: EAST CHINA NORMAL UNIVERSITY, Shanghai (CN)

(72) Inventors: Jian ZHOU, Shanghai (CN); BoShuai MU, Shanghai (CN); JinSheng YU, Shanghai (CN)

(73) Assignee: EAST CHINA NORMAL UNIVERSITY, Shanghai (CN)

(21) Appl. No.: 18/031,243

(22) PCT Filed: Nov. 1, 2021
(86) PCT No.: PCT/CN2021/127919

§ 371 Date: Apr. 11, 2023

(30) Foreign Application Priority Data
Nov. 6, 2020 (CN) 202011228303.0

(51) Int. Cl. C07F 7/18; C07F 7/08; C07F 7/12; C07D 453/04; C07C 33/18; C07D 215/12

(52) U.S. Cl. CPC C07F 7/1804; C07F 7/081; C07F 7/12; C07D 453/04; C07F 7/0834; C07C 33/18; C07D 215/12

(57) ABSTRACT

Disclosed in the present invention are a functionalized fluoroalkyl silane compound and a synthetic method therefor. The method comprises: dissolving a halosilane and a fluoroalkyl source in an organic solvent; and synthesizing functionalized fluoroalkyl silane under the effect of an alkali or a tertiary phosphine compound. The functionalized fluoroalkyl silane can not only be used for constructing a series of high added-value compounds such as fluoroalkyl substituted alcohols, ketones and amines that can be constructed by conventional TMSR_{f}, but also can transfer, by means of appropriate conversion, a functional group on a silicon protecting group to the obtained addition product in an addition reaction, for synthesizing some fluorine-containing compounds that cannot be synthesized by using a conventional TMSR_{f} reagent, thereby greatly improving the synthesis efficiency and the atom economy of reactions. Also disclosed in the present invention are more excellent reaction efficiency and enantioselectivity, compared with conventional TMSCF₃, exhibited by trifluoromethyl chloromethylsilane in synthesis of a 2-trifluoromethylquinoline compound and in an asymmetric trifluoromethylation reaction with α,β-unsaturated ketones.

### TECHNICAL FIELD

The invention belongs to the technical field of organic synthesis, and in particular relates to a functionalized fluoroalkyl silane and synthetic method thereof and application.

### BACKGROUND TECHNIQUE

Selective introduction of fluoroalkyl groups into organic compounds usually significantly changes the physical, chemical and biological activities of their parent compounds, and has the effect of improving the metabolic stability and bioavailability of bioactive molecules, thereby making the compounds with fluoroalkyl structure widely present in various drugs and related active compounds. For example: the anti-AIDS drug Efavirenz (Efavirenz), the anti-malarial drug Mefloquine ((+)-erythro-Mefloquine), the antidepressant drug Befloxatone (Befloxatone), the polio drug Afloqualone (Afloqualone), the antitumor drug Garenoxacin (Garenoxacin) and the antihypertensive drug KC-515 are all drugs containing this type of dominant structural unit, and the structures of the above drugs are shown below.

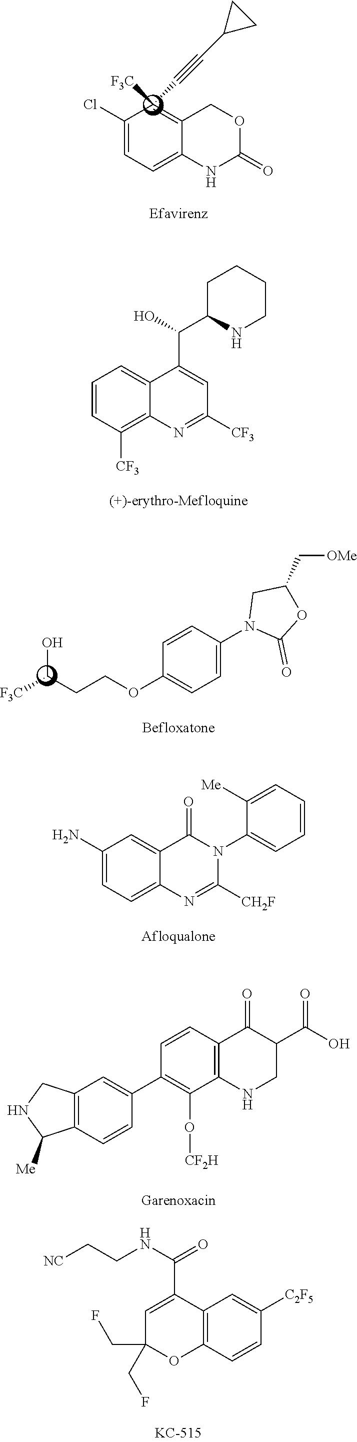

Among the methods for introducing trifluoromethyl groups, the nucleophilic trifluoromethylation reaction involving fluoroalkyl silane that are stable to both acid and water is the most direct and effective method, and has been widely used in the synthesis of various high value-added fluorinated alkyl substituted compounds such as alcohols, ketones or amines. Therefore, how to efficiently synthesize fluoroalkyl silane with structural diversity has always been a hot research topic for chemists. Taking (trifluoromethyl)trimethylsilane (TMSCF₃) as an example, common synthetic methods include:

1) In 1984, Ruppert reported the first synthesis of TMSCF₃. They found that under the effect of Hexaethylphosphorous triamide[(Et₂N)₃P], trimethylchlorosilane(TMSCl) and trifluorobromomethane (CF₃Br) can successfully generate TMSCF₃. Then in 1999, Prakash optimized the method and found that using benzonitrile as solvent, under nitrogen protection, TMSCF₃ could be prepared on a large scale with a yield of 75% at −78 to −30° C. The reaction process is shown in Formula (II), route1. (Ruppert, I. et al, Tetrahedron Lett. 1984, 25, 2195-2198; Prakash, G. K. S. et al, J. Org. Chem. 1991, 56, 984-989.)

2) In 1989, Pawelke et al. prepared TMSCF₃ by reacting trifluoroiodomethane (CF₃I) with TMSCl in the presence of tetrakis(dimethylamino)ethylene. It is found that TMSCF₃ can be obtained with a yield of up to 94% at −196° C., as shown in Formula (II), route2. It should be pointed out that the tetratris(dimethylamino)ethylene used in this method is relatively expensive, which is not conducive to the large-scale preparation and production of trifluoromethyl silane. (Pawelke, G. J. Fluorine Chem. 1989, 42, 429-433.)

3) In 2003, Prakash used chloroform (CF₃H) as the trifluoromethyl source, and first prepared it into the corresponding sulfonyl, sulfoxide or thioether oxatrifluoromethyl compounds, and then reacted with TMSCl under the effect of magnesium metal to synthesize the target TMSCF₃ in high yield, as shown in formula (II), route 3. Although this method uses cheaper and readily available chloroform as the trifluoromethyl source, but the step is less ecomomical, and the reaction generate unpleasant smelling sulfur-containing by-products. (Prakash, G. K. S. et al, J. Org. Chem. 2003, 68, 4457-4463.)

4) In 2012, Prakash et al. further optimized the method of synthesizing TMSCF₃ from CF₃H. After continuous exploration, it was found that TMSCF₃ was obtained by one step reaction of CF₃H and TMSCl in 80% yield using toluene as solvent under the effect of strong base potassium bis(trimethylsilyl)amide (KHMDS), as shown in formula (II), Route 4. This is a commonly used method for large-scale synthesis of TMSCF₃. (Prakash, G. K. S. et al, Science 2012, 338, 1324-1327.)

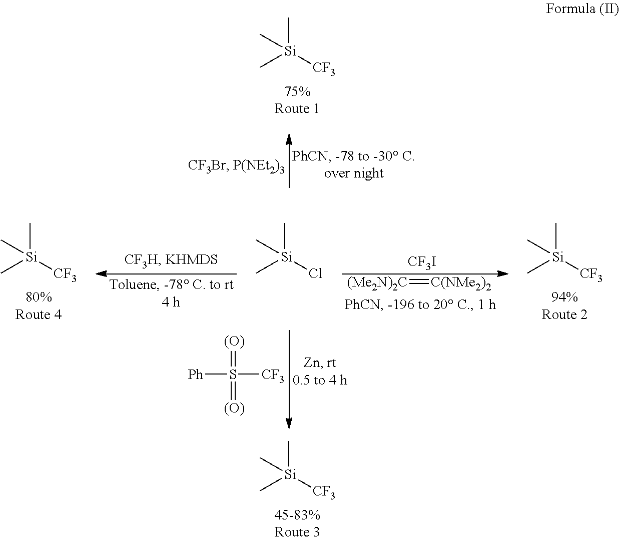

In summary, although several synthetic routes have been developed to prepare fluoroalkyl silane, the vast majority of these methods are only reported for the synthesis of simple fluoroalkyl silanes (R_{f}TMS). The synthesis of functionalized fluoroalkyl silane has not been reported in any literature so far.

### SUMMARY OF THE INVENTION

In order to solve the deficiencies in the prior art, the purpose of the present invention is to provide a series of high-purity novel functionalized fluoroalkyl silane compounds 2 with commercially available halosilane compound 1 and fluoroalkyl sources (R_{f}X) as raw materials, under the effect of cheap and easily available alkali or tertiary phosphine compounds (PR²₃) in high yield.

The present invention provides a method for synthesizing of functionalized fluoroalkyl silane compounds. In solvent, the fluoroalkyl source R_{f}X and halosilane compounds are used as raw materials to react under the effect of alkali or tertiary phosphine compounds (PR²₃) to obtain functionalized fluoroalkyl silane compounds.

The reaction scheme of the synthetic method of the present invention is shown in formula (I):

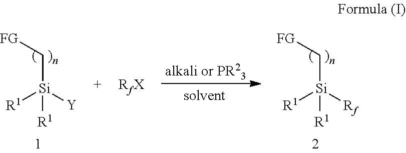

Wherein,

FG is halogen, OMs, OTs, NO₂, CF₃, CN, CO₂R, CONR₂, —CH═CR₂, —C≡CR, etc., R is H, C₁₋₁₀ alkyl, C₁₋₁₅ aromatic ring, thiophene, furan, pyrrole, pyridine, etc.;

R_{f} is a C₁₋₁₀ alkyl group containing fluorine atoms, etc.;

R¹ is C₁₋₁₀ alkyl, aryl, etc.;

The aryl group is the electron donating group substituted benzene ring, the electron withdrawing group substituted benzene ring, naphthyl, thiophene, furan, pyrrole, pyridine, ester group, etc.; wherein, the electron donating group includes C₁₋₁₀ alkyl, C₁₋₁₀ alkoxy, etc., the electron withdrawing group includes trifluoromethyl, ester group, nitro, cyano, halogen, etc.;

Y is halogen, OTf, etc;

n=1-10;

X is H, halogen, etc;

Preferably,

FG is F, Cl, Br, I, OMs, OTs, NO₂, CF₃, CN, CO₂R, CONR₂, —CH═CR₂, —C≡CR etc., R is H, C₁₋₁₀ alkyl, C₁₋₁₅ aromatic ring, thiophene, furan, pyrrole, pyridine, etc.;

R_{f} is CF₃, CF₂H, CFH₂, C₂F₅, CF₂CF₂H, CF₂CF₂Cl, CF₂CF₂Br, CF₂CH₃, C₃F₇, CF₂CF₂CF₂H, CF₂CF₂CH₃, CF₂CH₂CH₃, C₄F₉, CF₂CF₂CF₂CF₂H, CF₂CF₂CF₂CH₃, CF₂CF₂CH₂CH₃, CF₂CH₂CH₂CH₃, etc;

R¹ is C₁₋₁₀ alkyl group, electron donating group substituted benzene ring, electron withdrawing group substituted benzene ring, naphthyl, thiophene, furan, pyrrole, pyridine, ester group, etc.; wherein, the electron donating group includes methyl, methoxy, etc., the electron withdrawing group includes trifluoromethyl, ester group, nitro, cyano, fluorine, chlorine, bromine, iodine, etc.;

Y is Cl, Br, I, OTf, etc.;

n=1-10;

X is H, Br, I, etc.

Wherein, the alkali is one or more of the following: lithium bis(trimethylsilyl) amide (LiHMDS), potassium bis(trimethyl silyl) amide (KHMDS), sodium bis(trimethylsilyl) amide (NaHMDS), sodium amide (NaNH₂), sodium hydride (NaH), etc.; preferably, potassium bis(trimethylsilyl) amide (KHMDS).

Wherein, R² is C₁₋₁₀ alkyl group, C₁₋₁₀ alkoxy group, C₁₋₁₀ alkylamine group, aryl group, etc., and the aryl group is electron donating group substituted benzene ring, electron withdrawing group substituted benzene ring, naphthyl, thiophene, furan, pyrrole, pyridine, ester group, etc.; wherein, the electron donating group includes C₁₋₁₀ alkyl group, C₁₋₁₀ alkoxy group, etc., and the electron withdrawing group includes trifluoromethyl, ester group, nitro, cyano, halogen, etc.; preferably, C₁₋₁₀ alkylamine group.

Wherein, the reaction is preferably carried out under a nitrogen atmosphere.

Wherein, the temperature of the reaction is −78˜100° C.; preferably, the temperature is −78˜30° C.

Wherein, the reaction time is 2-36 hours; preferably, the time is 12 hours.

Wherein, the halosilane compound 1 is a commercially available raw material; R_{f}X is a reagent for providing a fluoroalkyl source.

Wherein, when the fluoroalkyl source is CF₃H, CF₂H2, HCF₂CH₃, HCF₂CH₂CH₃, HCF₂CH₂CH₂CH₃, the reaction is completed under the effect of alkali, and its effect is to grab the proton that takes the a position of the fluorine atom; when the fluoroalkyl source is XCF₃, XCF₂H, XCFH₂, XC₂F₅, XCF₂CF₂H, XCF₂CF₂Cl, XCF₂CF₂Br, XCF₂CH₃, XC₃F₇, XCF₂CF₂CF₂H, XCF₂CF₂CH₃, XCF₂CH₂CH₃, XC₄F₉, XCF₂CF₂CF₂CF₂H, XCF₂CF₂CF₂CH₃, XCF₂CF₂CH₂CH₃, XCF₂CH₂CH₂CH₃ (X=Br or I), the reaction is carried out under the effect of a tertiary phosphine compound (PR²₃), and its function is to activate the fluoroalkyl source.

Wherein, the molar ratio of the fluoroalkyl source R_{f}X, the halosilane compound and the alkali (or PR²₃) is R_{f}X: halosilane compound: alkali (or PR²₃)=(1-20):(1-3):(1-3); preferably, 3:1:1.2.

Wherein, the solvent is any one or more of the following: benzonitrile, phenylacetonitrile, acetonitrile, dichloromethane, toluene, tetrahydrofuran(THF), diethyl ether, dimethylformamide (DMF), dimethylacetamide, dimethyl sulfoxide (DMSO), N-methylpyrrolidone (NMP), hexamethyiphosphoric triamide (HMPA), etc.; preferably, any one or more of benzonitrile, toluene, tetrahydrofuran (THF).

Wherein, the novel functionalized fluoroalkyl silane compound (silyl fluoroalkylation reagent 2) is the target product of the synthesis method of the present invention.

The present invention also provides functionalized fluoroalkyl silane compounds, the structure of which is shown in formula (1):

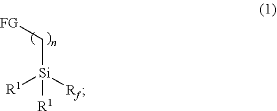

Wherein,

FG is halogen, OMs, OTs, NO₂, CF₃, CN, CO₂R, CONR₂, —CH═CR₂, —C≡CR, etc., R is H, C₁₋₁₀ alkyl, C₁₋₁₅ aromatic ring, thiophene, furan, pyrrole, pyridine, etc.;

R_{f} is a C₁₋₁₀ alkyl group containing fluorine atoms, etc.;

R₁ is C₁₋₁₀ alkyl, aryl, etc.;

The aryl group is the electron donating group substituted benzene ring, the electron withdrawing group substituted benzene ring, naphthyl, thiophene, furan, pyrrole, pyridine, ester group, etc.; wherein, the electron donating group includes C₁₋₁₀ alkyl, C₁₋₁₀ alkoxy, etc., the electron withdrawing group includes trifluoromethyl, ester group, nitro, cyano, fluorine, chlorine, bromine, iodine, etc.;

n=1-10;

Preferably,

FG is F, Cl, Br, I, OMs, OTs, NO₂, CF₃, CN, CO₂R, CONR₂, —CH═CR₂, —C≡CR, Wherein R is H, C₁₋₁₀ alkyl, C₁₋₁₅ aromatic ring, thiophene, furan, pyrrole, pyridine;

R_{f} is CF₃, CF₂H, CFH₂, C₂F₅, CF₂CF₂H, CF₂CF₂Cl, CF₂CF₂Br, CF₂CH₃, C₃F₇, CF₂CF₂CF₂H, CF₂CF₂CH₃, CF₂CH₂CH₃, C₄F₉, CF₂CF₂CF₂CF₂H, CF₂CF₂CF₂CH₃, CF₂CF₂CH₂CH₃, CF₂CH₂CH₂CH₃;

R₁ is C₁₋₁₀ alkyl group, electron donating group substituted benzene ring, electron withdrawing group substituted benzene ring, naphthyl, thiophene, furan, pyrrole, pyridine, ester group, etc.; wherein, the electron donating group includes methyl, methoxy, the electron withdrawing group includes trifluoromethyl, ester group, nitro, cyano, fluorine, chlorine, bromine, iodine, etc.;

n=1-10.

The present invention also provides the application of the functionalized fluoroalkyl silane compounds in the silylation reaction and the functional group transfer reaction.

The present invention also provides a method of using the functionalized fluoroalkyl silane compounds in several types of addition reactions, and further the functional group on the silicon protecting group is transferred to the obtained addition products through appropriate transformation, therefore, the synthesis efficiency and the atom economy of the reaction are greatly improved. For representative examples, please refer to application examples 1-4.

In the silico-fluoroalkylation, the quinine-derived chiral phase transfer catalyst, TMAF, toluene and dichloromethane mixed solvent are added to the raw material in a dry Schlenk tube, and the resulting mixed solution is added to the functionalized fluoroalkyl silane compounds prepared by the method of the present invention at low temperature after stirring. Then, the reaction process is monitored by thin layer chromatography. After the raw materials are consumed, column chromatography is directly performed and then measure the yield. The subsequent functional group transfer can be achieved by free radical reactions.

The advantages of the present invention are: all kinds of reagents used in the present invention are commercially available, the raw materials are from a wide range of sources, with low prices, and the various reagents can exist stably under normal temperature and pressure, and the operation and handling are convenient; there is no special requirement for equipment and no special requirement for postprocessing; the functionalized fluoroalkyl silane compounds synthesized in the present invention have broad application prospects. The functionalized fluoroalkyl silane compounds involved in the silico-fluoroalkylation reaction can not only be used for constructing fluoroalkyl substituted alcohols, fluoroalkyl substituted ketones and α-fluoroalkyl substituted amines and other important fluoroalkyl intermediates that can be constructed by conventional TMSR_{f}, but also can transfer, by means of appropriate conversion, a functional group on a silicon protecting group to the obtained addition product in an addition reaction, for synthesizing some fluorine-containing compounds that cannot be synthesized by using a conventional TMSR_{f}, thereby greatly improving the synthesis efficiency and enantioselectivity.

### PREFERRED EMBODIMENTS OF THE INVENTION

The following examples are given for the further illustration of the present invention. The process, conditions, experimental methods, and so on for implementing the present invention are all general knowledge and common knowledge in the field except for the contents specifically mentioned below, and the present invention has no special limitation.

### EXAMPLES

### Synthesis of Functionalized Fluoroalkyl Silane Compounds

### 1) Conversion from Compound 1aa-1ad to Compound 2a

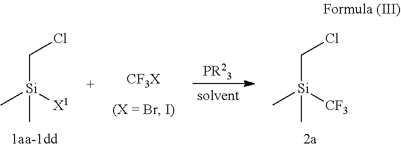

General operation procedure 1: CF₃X (150-300 mmol) was condensated into a dry 250 mL three-necked flask at −78° C., and the organic solvent (80 mL), freshly distilled of halosilane 1aa-1ad (50-300 mmol) and tertiary phosphine (PR²₃) (50-300 mmol) were slowly added to the reaction flask at this temperature; the resulting mixed solution was slowly raised to the temperature shown in Table 1 and stirred for reaction. The reaction process was monitored by ¹H NMR. After the raw materials 1aa-1ad were consumed, 2a as shown in Formula (III) was obtained by distillation under reduced pressure.

The specific experimental operations of Examples 1-15 are shown in general operation procedure 1, and the specific reaction condition and yield of each example are shown in Table 1.

TABLE 1
Specific reaction conditions and yields of specific Examples 1-15

| | 1 (X) (mmol)/CF₃X (mmoL)/PR²₃ | | Temperature | Time | Yield |
|---|---|---|---|---|---|
| Example | (mmol) | Solvent | (° C.) | (h) | (%) |
| 1 | 1aa Cl (150)/CF₃Br (300)/P(NEt₂)₃ | PhCN | −30° C. | 12 | 78 |
| | (150) | | | | |
| 2 | 1aa Cl (100)/CF₃Br (200)/P(NEt₂)₃ | PhCN | −50° C. | 12 | 75 |
| | (100) | | | | |
| 3 | 1aa Cl (100)/CF₃Br (150)/P(NEt₂)₃ (80) | PhCN | −60° C. | 12 | 56 |
| 4 | 1aa Cl (150)/CF₃Br (300)/P(OEt₂)₃ | Toluene | −78° C. | 12 | 42 |
| | (150) | | | | |
| 5 | 1aa Cl (150)/CF₃Br (300)/P(OMe)₃ | THF | −78° C. | 12 | 46 |
| | (150) | | | | |
| 6 | 1aa Cl (150)/CF₃Br (300)/P(C₃H₇)₃ | PhCN | −78° C. | 6 | 36 |
| | (150) | | | | |
| 7 | 1aa Cl (150)/CF₃Br (300)/P(C₂H₅)₃ | PhCN | −60° C. | 12 | 34 |
| | (150) | | | | |
| 8 | 1aa Cl (100)/CF₃I (200)/P(NEt₂)₃ (100) | PhCN | −78° C. | 12 | 60 |
| 9 | 1aa Cl (100)/CF₃I (200)/P(NEt₂)₃ (100) | PhCN | −50° C. | 12 | 63 |
| 10 | 1aa Cl (100)/CF₃I (200)/P(NEt₂)₃ (100) | PhCN | −50° C. | 6 | 59 |
| 11 | 1aa Cl (100)/CF₃Br (200)/P(NMe₂)₃ | PhCN | −78° C. | 12 | 40 |
| | (100) | | | | |
| 12 | 1aa Cl (150)/CF₃Br (300)/P(NMe₂)₃ | PhCN | −78° C. | 12 | 35 |
| | (150) | | | | |
| 13 | 1ab Br (150)/CF₃Br (300)/P(NEt₂)₃ | PhCN | −78° C. | 12 | 54 |
| | (150) | | | | |
| 14 | 1ac I (150)/CF₃Br (300)/P(NEt₂)₃ (150) | PhCN | −78° C. | 12 | 45 |
| 15 | 1ad I (150)/CF₃Br (300)/P(NEt₂)₃ (150) | PhCN | −50° C. | 8 | 53 |

The NMR characterization data for compound 2a are as follows:

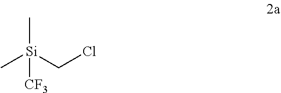

¹H NMR (400 MHz, CDCl₃): δ 2.97 (s, 2H), 0.42 (s, 6H); ¹³C NMR (100 MHz, CDCl₃): δ 130.4 (q, J=319 Hz), 25.3, −7.8; ¹⁹F NMR (376 MHz, CDCl₃): δ −64.31 (s, 3F).

2) Conversion from Compound 1b-1c to Compound 2b-2e

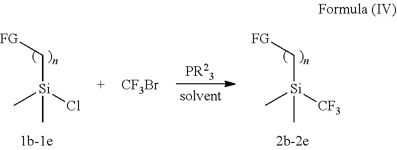

General operation procedure 2: CF₃Br (300 mmol) was condensated into a dry 250 mL three-necked flask at −78° C., and the organic solvent (80 mL), freshly distilled of halosilane 1b-1e (150 mmol) and PR²₃ (150 mmol) were slowly added to the reaction flask at this temperature: the resulting mixed solution was slowly raised to the temperature shown in Table 2 and stirred for reaction. The reaction process was monitored by ¹H NMR. After the raw materials 1b-1e were consumed, 2b-2e as shown in Formula (IV) were obtained by distillation under reduced pressure.

The specific experimental operations of Examples 16-34 are shown in general operation procedure 2, and the specific reaction condition and yield of each example are shown in Table 2.

TABLE 2
Specific reaction conditions and yields of specific Examples 16-34

| | | | | Temperature | Time | Product/Yield |
|---|---|---|---|---|---|---|
| Example | 1b-1e | PR²₃ | Solvent | (° C.) | (h) | (%) |
| 16 | 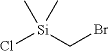 | P(NEt₂)₃ | PhCN | −30 | 12 | 2b/62 |
| | 1b | | | | | |
| 17 | 1b | P(NMe₂)₃ | PhCH₂CN | −60 | 12 | 2b/41 |
| 18 | 1b | P(OMe₂)₃ | Et₂O | −78 | 10 | 2b/52 |
| 19 | 1b | P(OEt₂)₃ | THF | −78 | 8 | 2b/55 |
| 20 | 1b | P(C₂H₅)₃ | CH₃CN | −78 | 7 | 2b/47 |
| 21 | 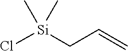 | P(NEt₂)₃ | PhCN | −30 | 12 | 2c/70 |
| | 1c | | | | | |
| 22 | 1c | P(NMe₂)₃ | PhCH₂CN | −60 | 12 | 2c/52 |
| 23 | 1c | P(OMe₂)₃ | Et₂O | −78 | 12 | 2c/38 |
| 24 | 1c | P(OEt₂)₃ | THF | −78 | 10 | 2c/62 |
| 25 | 1c | P(C₂H₅)₃ | CH₃CN | −78 | 12 | 2c/66 |
| 26 | 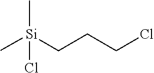 | P(NEt₂)₃ | PhCN | −30 | 12 | 2d/60 |
| | 1d | | | | | |
| 27 | 1d | P(NMe₂)₃ | PhCH₂CN | −60 | 10 | 2d/47 |
| 28 | 1d | P(OMe₂)₃ | Et₂O | −78 | 8 | 2d/56 |
| 29 | 1d | P(OEt₂)₃ | THF | −78 | 12 | 2d/50 |
| 30 | 1d | P(C₂H₅)₃ | CH₃CN | −78 | 12 | 2d/46 |
| 31 | 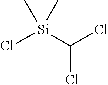 | P(NEt₂)₃ | PhCN | −30 | 20 | 2e/53 |
| | 1e | | | | | |
| 32 | 1e | P(NMe₂)₃ | PhCH₂CN | −60 | 12 | 2e/59 |
| 33 | 1e | P(OMe₂)₃ | Et₂O | −78 | 16 | 2e/47 |
| 34 | 1e | P(OEt₂)₃ | THF | −78 | 20 | 2e/41 |

The NMR characterization data for compound 2b-2e are as follows:

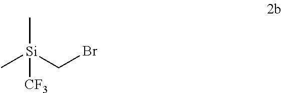

¹H NMR (400 MHz, CDCl₃): δ 2.75 (s, 2H), 0.39 (s, 6H): ¹³C NMR (100 MHz, CDCl₃): δ 130.8 (q, J=315 Hz), 27.0, −6.4; ¹⁹F NMR (376 MHz, CDCl₃): δ −64.73 (s, 3F).

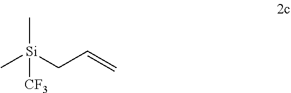

¹H NMR (400 MHz, CDCl₃): 5.72 (m, 1H), 5.03-5.10 (m, 2H), 1.67 (d, J=8.0 Hz, 2H), 0.28 (s, 6H); ¹³C NMR (100 MHz, CDCl₃): δ 135.2, 132.1 (q, J=311 Hz), 119.4, 11.2, −5.6; ¹⁹F NMR (376 MHz, CDCl₃): δ −65.56 (s, 3F).

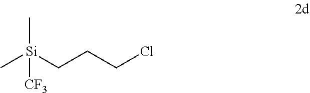

¹H NMR (400 MHz, CDCl₃): δ 3.85 (t, J=8.0 Hz, 2H), 1.49-1.63 (m, 2H), 1.17 (t, J=8.0 Hz, 2H), 0.43 (s, 6H); ¹³C NMR (100 MHz, CDCl₃): δ 132.0 (q, J=322 Hz), 47.2, 27.6, 1.4, −5.4; ¹⁹F NMR (376 MHz, CDCl₃): δ −66.78 (s, 3F).

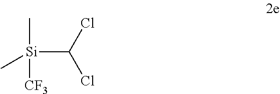

¹H NMR (400 MHz, CDCl₃): δ 5.28 (s, 1H), 0.59 (s, 6H); ¹³C NMR (100 MHz, CDCl₃): δ 141.3 (q, J=325 Hz), 31.2, −3.5; ¹⁹F NMR (376 MHz, CDCl₃): δ −62.54 (s, 3F).

3) Conversion from Compound 1Aa to Compound 2f-2i

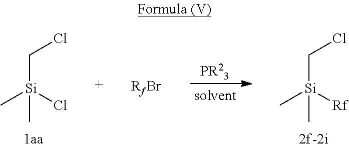

General operation procedure 3: R_{f}Br (300 mmol) was condensated into a dry 250 mL three-necked flask at −78° C., and the organic solvent (80 mL), freshly distilled of halosilane 1aa (150 mmol) and PR²₃ (150 mmol) were slowly added to the reaction flask at this temperature; the resulting mixed solution was slowly raised to the temperature shown in Table 3 and stirred for reaction. The reaction process was monitored by ¹H NMR. After the raw material 1aa was consumed, 2f-2i as shown in Formula (V) were obtained by distillation under reduced pressure.

The specific experimental operations of Examples 35-45 are shown in general operation procedure 3, and the specific reaction condition and yield of each example are shown in Table 3.

TABLE 3
Specific reaction conditions and yields of specific Examples 35-45

| | | | | | | Prod- |
|---|---|---|---|---|---|---|
| | | | | Temper- | | uct/ |
| Exam- | | | | ature | Time | Yield |
| ple | R_{f}Br | PR²₃ | Solvent | (° C.) | (h) | (%) |
| 35 | BrCF₂H | P(NEt₂)₃ | PhCN | −50 | 8 | 2f/68 |
| 36 | BrCF₂H | P(OEt₂)₃ | Et₂O | −100 | 8 | 2f/43 |
| 37 | BrCF₂H | P(C₂H₅)₃ | MeCN | −78 | 10 | 2f/44 |
| 38 | BrCF₂CF₃ | P(NEt₂)₃ | PhCN | −50 | 13 | 2g/70 |
| 39 | BrCF₂CF₃ | P(OEt₂)₃ | Et₂O | −78 | 12 | 2g/67 |
| 40 | BrCF₂CF₃ | P(C₂H₅)₃ | THF | −78 | 12 | 2g/43 |
| 41 | BrCF₂CF₂CF₃ | P(NEt₂)₃ | PhCN | −50 | 5 | 2h/45 |
| 42 | BrCF₂CF₂CF₃ | P(OEt₂)₃ | Et₂O | −50 | 12 | 2h/56 |
| 43 | BrCF₂CF₂CF₃ | P(C₂H₅)₃ | THF | −78 | 12 | 2h/59 |
| 44 | BrCF₂CF₂H | P(NEt₂)₃ | PhCN | −50 | 7 | 2i/43 |
| 45 | BrCF₂CF₂H | P(OEt₂)₃ | Et₂O | −78 | 10 | 2i/39 |

The NMR characterization data for compound 2f-2i are as follows:

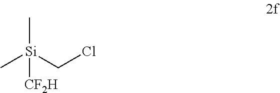

¹H NMR (400 MHz, CDCl₃): δ 5.27 (t, J=58.5 Hz, 1H), 2.89 (s, 2H), 0.35 (s, 6H); ¹³C NMR (100 MHz, CDCl₃): δ 110.4 (t, J=285 Hz), 20.4, −5.3; ¹⁹F NMR (376 MHz, CDCl₃): δ −140.33 (s, 2F).

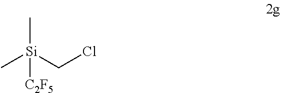

¹H NMR (400 MHz, CDCl₃): δ 3.39 (s, 2H), 0.68 (s, 6H); ¹³C NMR (100 MHz, CDCl₃): δ 145.1 (q, J=327.2 Hz), 113.5 (t, J=265.8 Hz), 28.4, −4.1; ¹⁹F NMR (376 MHz, CDCl₃): δ −129.35 (s, 2F): −80.45 (s, 3F).

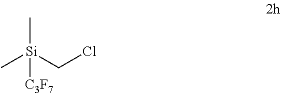

¹H NMR (400 MHz, CDCl₃): δ 3.41 (s, 2H), 0.75 (s, 6H); ¹³C NMR (100 MHz, CDCl₃): δ 145.8 (q, J=322.5 Hz), 117.6 (t, J=262.8 Hz), 113.5 (t, J=255.8 Hz), 28.4, −4.1; ¹⁹F NMR (376 MHz, CDCl₃): δ −129.06 (s, 2F), −125.33 (s, 2F), −79.45 (s, 3F).

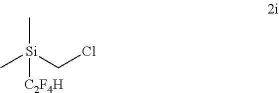

¹H NMR (400 MHz, CDCl₃): δ 5.47 (t, J 56.5 Hz, 1H), 2.91 (s, 2H), 0.35 (s, 6H); ¹³C NMR (100 MHz, CDCl₃): δ 111.4 (t, J 280 Hz), 109.3 (t, J=256 Hz), 20.4, −5.3; ¹⁹F NMR (376 MHz, CDCl₃): δ −130.12 (s, 2F), −138.42 (s, 2F).

3) Conversion from Compound 1a-1c to Compound 2a-2c, 2f

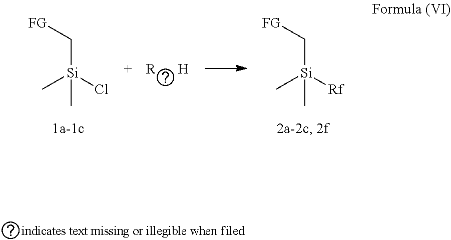

General operation procedure 4: alkali (100-300 mmol) was added into a dry 250 mL three-necked flask, freshly distilled of halosilane 1a-1c (100-300 mmol) was added at −78 C, then R_{f}H (100-300 mmol) gas was added into the low-temperature reaction system (bubbling for 2 h), the resulting mixed solution was stirred for reaction at the temperature shown in Table 4. The reaction process was monitored by ¹H NMR. After the raw materials 1a-1c were consumed, 2a-2c or 2f as shown in Formula (VI) were obtained by distillation under reduced pressure.

The specific experimental operations of Examples 46-61 are shown in general operation procedure 4, and the specific reaction condition and yield of each example are shown in Table 4.

TABLE 4
Specific reaction conditions and yields of specific examples 46-61

| | | | | | Product/ |
|---|---|---|---|---|---|
| | 1a-1c (FG) (mmol) /R_{f}H (mmol)/alkali | | Temperature | Time T | Yield |
| Example | (mmol) | Solvent | (° C.) | (h) | (%) |
| 46 | 1a (Cl) (280)/CF₃H (225)/KHMDS | Toluene | −78 | 7 | 2a/70 |
| | (225) | | | | |
| 47 | 1a (Cl) (280)/CF₃H (280)/LiHMDS | THF | −50 | 5 | 2a/68 |
| | (280) | | | | |
| 48 | 1a (Cl) (280)/CF₃H (300)/NaHMDS | Toluene | −78 | 8 | 2a/54 |
| | (280) | | | | |
| 49 | 1a (Cl) (100)/CF₃H (225)/NaH (100) | DMF | −78 | 10 | 2a/23 |
| 50 | 1b (Br) (280)/CF₃H (300)/KHMDS | Toluene | −78 | 7 | 2b/59 |
| | (225) | | | | |
| 51 | 1b (Br) (280)/CF₃H (280)/LiHMDS | THF | −50 | 5 | 2b/67 |
| | (280) | | | | |
| 52 | 1b (Br) (280)/CF₃H (300)/NaHMDS | Toluene | −78 | 8 | 2b/44 |
| | (280) | | | | |
| 53 | 1b (Br) (100)/CF₃H (225)/NaH (100) | DMF | −78 | 10 | 2b/31 |
| 54 | 1c (CH═CH₂) (280)/CF₃H (225)/ | Toluene | −78 | 12 | 2c/77 |
| | KHMDS (225) | | | | |
| 55 | 1c (CH═CH₂) (280)/CF₃H (300)/ | THE | −50 | 12 | 2c/73 |
| | LiHMDS (280) | | | | |
| 56 | 1c (CH═CH₂) (280)/CF₃H (300)/ | Toluene | −78 | 15 | 2c/45 |
| | NaHMDS (280) | | | | |
| 57 | 1c (CH═CH₂) (100)/CF₃H (225)/NaH | DMF | −78 | 15 | 2c/38 |
| | (100) | | | | |
| 58 | 1a (Cl) (100)/CF₂H₂ (225)/KHMDS | Toluene | −100 | 8 | 2f/70 |
| | (100) | | | | |
| 59 | 1a (Cl) (280)/CF₂H₂ (280)/LiHMDS | THF | −100 | 12 | 2f/56 |
| | (150) | | | | |
| 60 | 1a (Cl) (280)/CF₂H₂ (300)/NaHMDS | Toluene | −81 | 6 | 2f/45 |
| | (150) | | | | |
| 61 | 1a (Cl) (100)/CF₂H₂ (225)/NaH (100) | DMF | −50 | 7 | 2f/28 |

### Application of Functionalized Fluoroalkyl Silane

### Application Example 1: Asymmetric Trifluoromethylation Reaction Involving the Functionalized Trifluoromethyl Silane 2a Synthesized in Example 2 of the Present Invention, Follows the Reaction Path of Formula (VII)

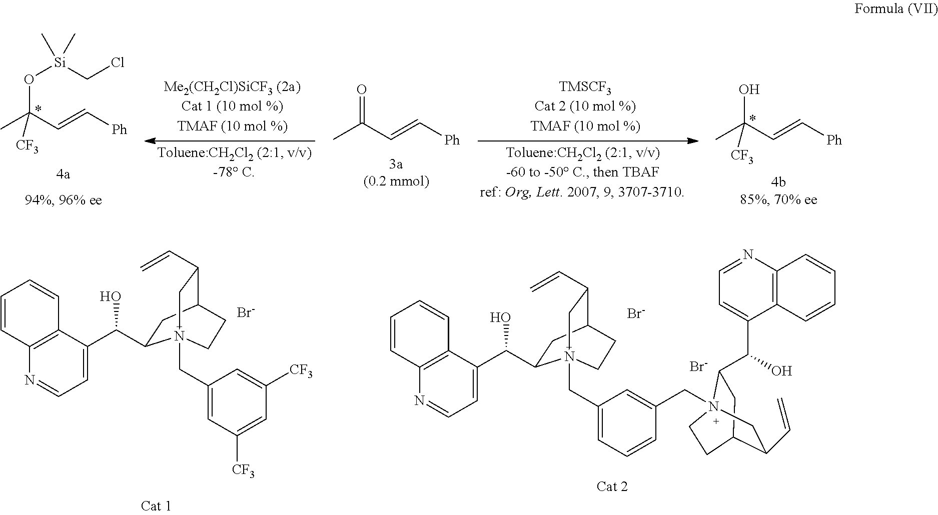

In a dry 25 mL Schlenk tube, under the protection of nitrogen were added raw material 3a (29 mg, 0.2 mmol), Cat 1 (12 mg, 0.02 mmol), TMAF (2 mg, 0.02 mmol), followed by a mixed solution of anhydrous toluene and anhydrous dichloromethane (2.0 mL) with a volume ratio of 2:1, the resulting mixed solution was stirred at −78° C. for 10 min, and then 2a (70 μL, 0.4 mmol) was added to react. The reaction process was monitored by thin-layer chromatography. After the raw material 3a was consumed, 4a as shown in Formula (VII) was obtained by direct column chromatography with a yield of 94%.

The relevant characterization data for compound 4a are as follows:

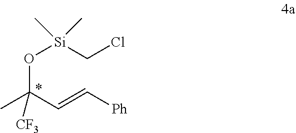 [SMILES: CC(/C=C/c1ccccc1)(O[Si](C)(C)CCl)C(F)(F)F]

HPLC analysis: Chiralcel OJ-H, isopropanol/n-hexane=0.5/99.5, 10 mL/min, 230 nm; tᵣ (major)=6.62 min, tᵣ (minor)=8.03 min, 96% ee;

Optical rotation: [α]_{D}²⁵=+38.6 (c=1.0, CHCl₃);

¹H NMR (300 MHz, CDCl₃): 7.44-7.31 (m, 5H), 6.90 (d, J=8.0 Hz, 1H), 6.35 (d, J=8.0 Hz, 1H), 3.96 (s, 3H), 2.96 (s, 2H), 0.40 (d, J=2.0 Hz, 6H); ¹³C NMR (100 MHz, CDCl₃): δ 137.3, 134.5, 130.8, 129.5, 128.8, 127.7 (q, J=287 Hz), 126.28, 75.8 (q, J=29 Hz), 29.8, 22.34, 3.3; ¹⁹F NMR (376 MHz, CDCl₃): δ −78.34 (s, 3F).

### Application Example 2: Asymmetric Trifluoromethylation Reaction Involving the Functionalized Trifluoromethyl Silane 2a Synthesized in Example 2 of the Present Invention, Follows the Reaction Path of Formula (VIII)

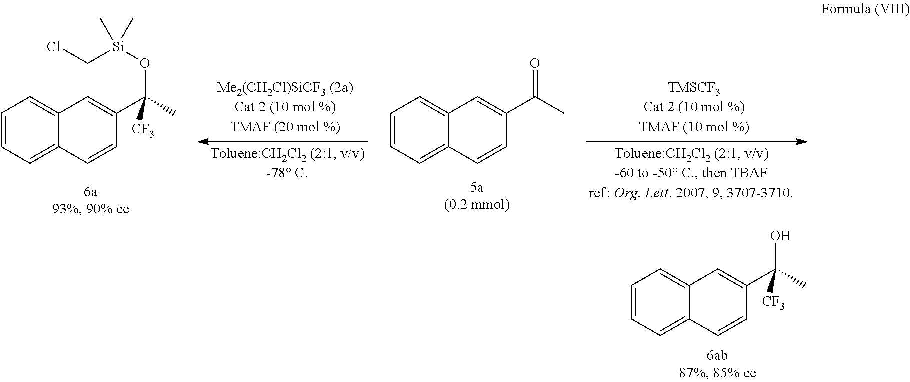 [SMILES: C[C@@](O[Si](C)(C)CCl)(c1ccc2ccccc2c1)C(F)(F)F ; CC(=O)c1ccc2ccccc2c1 ; C[C@@](O)(c1ccc2ccccc2c1)C(F)(F)F]

In a dry 25 mL Schlenk tube, under the protection of nitrogen were added raw material 5a (34 mg, 0.2 mmol), Cat 2 (17 mg, 0.02 mmol), TMAF (4 mg, 0.04 mmol), followed by a mixed solution of anhydrous toluene and anhydrous dichloromethane (2.0 mL) with a volume ratio of 2:1, the resulting mixed solution was stirred at −78° C. for 10 min, and then 2a (70 μL, 0.4 mmol) was added to react. The reaction process was monitored by thin-layer chromatography. After the raw material 5a was consumed, 6a as shown in Formula (VIII) was obtained by direct column chromatography with a yield of 93%.

The relevant characterization data for compound 6a are as follows:

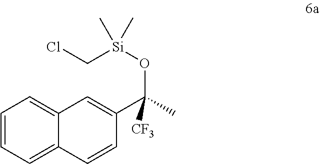 [SMILES: C[C@@](O[Si](C)(C)CCl)(c1ccc2ccccc2c1)C(F)(F)F]

HPLC analysis: Chiralcel OJ-H, isopropanol/n-hexane=0.5/99.5, 1.0 mL/min, 205 nm; tᵣ (major)=5.12 min, tᵣ (minor)=5.95 min, 90% ee;

Optical rotation: [α]_{D}²⁵=+8.3 (c=1.0, CHCl₃);

¹H NMR (400 MHz, CDCl₃): 7.99 (s, 1H), 7.87-7.82 (m, 3H), 7.65 (d, J=8.0 Hz, 1H), 7.52-7.48 (m, 2H), 2.80 (s, 2H), 1.94 (s, 3H), 0.28 (d, J=3.6 Hz, 6H); ¹³C NMR (100 MHz, CDCl₃): δ 128.6, 128.0, 127.6, 126.8, 126.5, 126.4, 125.3 (q, J=284 Hz), 124.4, 77.8 (q, J=29 Hz); ¹⁹F NMR (376 MHz, CDCl₃): δ −80.98 (s, 3F).

### Application Example 3: Trifluoromethylation Reaction of Quinoline Involving the Functionalized Trifluoromethyl Silane 2a Synthesized in Example 2 of the Present Invention, Follows the Reaction Path of Formula (IX)

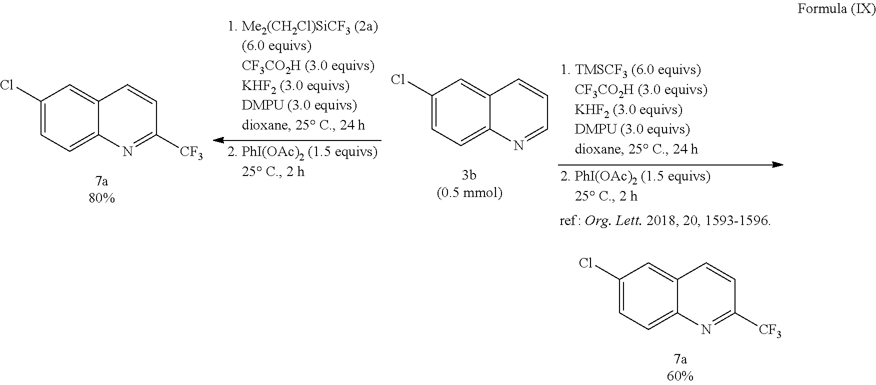 [SMILES: FC(F)(F)c1ccc2cc(Cl)ccc2n1 ; Clc1ccc2ncccc2c1 ; FC(F)(F)c1ccc2cc(Cl)ccc2n1]

In a plastic reaction tube that can be sealed with a stopcock, raw material 3b (82 mg, 0.5 mmol), KHF₂ (117 mg, 1.5 mmol), DMPU (189 mg, 1.5 mmol), 1,4-dioxane (5 mL) were added, followed by trifluoroacetic acid (170 mg, 1.5 mmol), the resulting mixed solution was stirred at 25° C. for 24 h, and 2a (528 μL, 3.0 mmol) was added to react, stirred at 25° C. for 24 h, then PhI(OAc)₂ (240 mg, 0.75 mmol) was added and stirred for 2 h. The reaction was quenched by addition of saturated sodium carbonate solution, extracted with ethyl acetate (10 ml x 6 times), the organic phases were combined, dried over anhydrous sodium sulfate, and the solvent was removed under reduced pressure. After purification by column chromatography, 7a of Formula (IX) can be obtained with a yield of 80%.

The relevant characterization data for compound 7a are as follows:

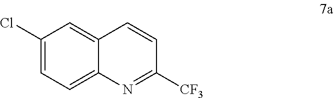 [SMILES: FC(F)(F)c1ccc2cc(Cl)ccc2n1]

¹H NMR (400 MHz, CDCl₃): δ 7.75 (d, J=8.5 Hz, 2H), 7.90 (s, 1H), 8.16 (d, J=9.0 Hz, 1H), 8.28 (d, J=8.5 Hz, 1H); ¹³C NMR (100 MHz, CDCl₃): δ 117.9 (q, J=2.2 Hz), 121.5 (q, J=275 Hz), 126.4, 129.5, 131.9, 132.1, 134.9, 137.4, 145.7, 148.4 (q, J=35.1 Hz); ¹⁹F NMR (376 MHz, CDCl₃): δ −69.5 (s, 3F).

### Application Example 4: The Functional Group Transfer Reaction Involving the Functionalized Trifluoromethyl Silane 4a Synthesized in Application Example 1 of the Present Invention, Follows the Reaction Path of Formula (X)

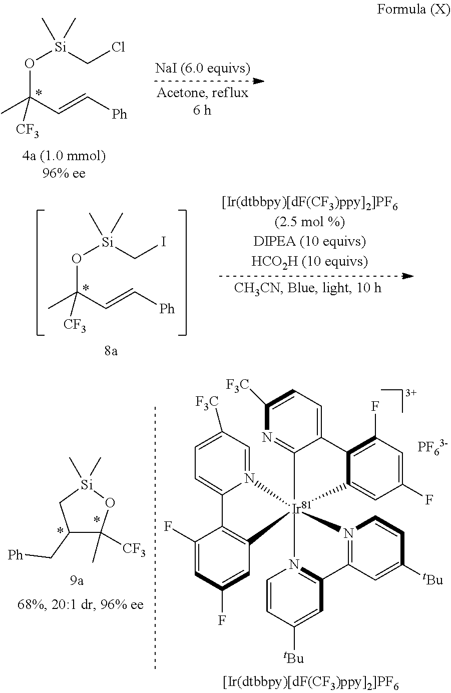

In a dry 25 mL round-bottomed flask, 4a (322 mg, 1.0 mmol), NaI (900 mg, 6.0 mmol) anhydrous acetone (10 mL) were added, the resulting solution was heated and stirred under reflux for 6 h, then a large amount of white solid (NaCl) was produced, the white solid was filtered off through silica gel and the solvent was removed from the filtrate under reduced pressure to afford 8a. In a dry 25 mL Schlenk tube, crude 8a and acetonitrile (10 mL) were added, followed by a mixed solution of diisopropylethylamine (1.30 g, 10 mmol) and formic acid (460 mg, 10 mmol), deoxygenated by nitrogen bubbling, followed by adding [Ir(dtbbpy)[dF(CF₃)ppy]₂]PF₆ (28 mg, 0.025 mmol), and then the reaction system was placed under blue light irradiation and stirred at room temperature for 10 h. After column chromatography, 9a as shown in Formula (X) can be obtained with a yield of 68% and dr value of 20:1.

The relevant characterization data for compound 9a are as follows:

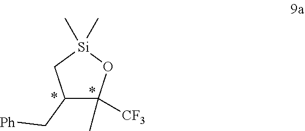 [SMILES: CC1(C(F)(F)F)O[Si](C)(C)CC1Cc1ccccc1]

HPLC analysis: Chiralcel OD-H, isopropanol/n-hexane=0.2/99.8, 1.0 mL/min, 205 nm; tᵣ (major)=7.86 min, tᵣ (minor)=8.58 min, 96% ee;

Optical rotation: [α]_{D}²⁵=+32.5 (c=1.0, CHCl₃);

¹H NMR (400 MHz, CDCl₃): 7.32-7.24 (m, 2H), 7.24-7.16 (m, 2H), 3.12-3.08 (m, 1H), 2.50-2.44 (m, 1H), 2.28 (t, J=12.0 Hz, 1H), 1.33 (s, 3H), 0.94-0.88 (m, 1H), 0.60-0.54 (m, 1H), 0.28 (s, 3H), 0.13 (s, 3H); ¹³C NMR (100 MHz, CDCl₃): δ 140.6, 129.1, 128.5, 126.9 (q, J=283 Hz), 126.3, 82.36 (q, J=28 Hz), 43.6, 39.8, 17.6, 16.8, 0.6, 0.4; ¹⁹F NMR (376 MHz, CDCl₃): δ−81.12 (s, 3F).

The protection content of the present invention is not limited to the above embodiments. Variations and advantages that can occur to those skilled in the art without departing from the spirit and scope of the inventive concept are included in the present invention, and the appended claims are the scope of protection.

1. A functionalized fluoroalkyl silane compound, wherein, the structure of the compound is shown in formula (1): 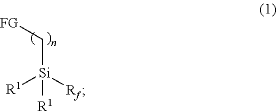
Wherein,
FG is halogen, OMs, OTs, NO₂, CF₃, CN, CO₂R, CONR₂, —CH═CR₂, —C≡CR, wherein, R is H, C₁₋₁₀ alkyl, C₁₋₁₅ aromatic ring, thiophene, furan, pyrrole, pyridine;
R_{f} is a C₁₋₁₀ alkyl group containing fluorine atoms;
R¹ is C₁₋₁₀ alkyl, aryl, the aryl is the electron donating group substituted benzene ring, the electron withdrawing group substituted benzene ring, naphthyl, thiophene, furan, pyrrole, pyridine, ester group; wherein, the electron donating group includes C₁₋₁₀ alkyl, C₁₋₁₀ alkoxy, the electron withdrawing group includes trifluoromethyl, ester group, nitro, cyano, halogen;
n=1-10.

2. The functionalized fluoroalkyl silane compound according to claim 1, wherein, FG is F, Cl, Br, I, OMs, OTs, NO₂, CF₃, CN, CO₂R, CONR₂, —CH═CR₂, —C≡CR, wherein, the R is H, C₁₋₁₀ alkyl, C₁₋₁₅ aromatic ring, thiophene, furan, pyrrole, pyridine; R_{f} is CF₃, CF₂H, CFH₂, C₂F₅, CF₂CF₂H, CF₂CF₂Cl, CF₂CF₂Br, CF₂CH₃, C₃F₇, CF₂CF₂CF₂H, CF₂CF₂CH₃, CF₂CH₂CH₃, C₄F₉, CF₂CF₂CF₂CF₂H, CF₂CF₂CF₂CH₃, CF₂CF₂CH₂CH₃, CF₂CH₂CH₂CH₃; R¹ is C₁₋₁₀ alkyl, electron donating group substituted benzene ring, electron withdrawing group substituted benzene ring, naphthyl, thiophene, furan, pyrrole, pyridine, ester group; wherein, the electron donating group includes methyl, methoxy, the electron withdrawing group includes trifluoromethyl, ester group, nitro, cyano, fluorine, chlorine, bromine, iodine; n=1-10.

3. A synthesis method of functionalized fluoroalkyl silane compound, wherein, the fluoroalkyl source R_{f}X reacts with halosilane compound in solvent under the effect of alkali or tertiary phosphine compound PR²₃ to obtain functionalized fluoroalkyl silane compound; the reaction scheme is shown in formula (I): 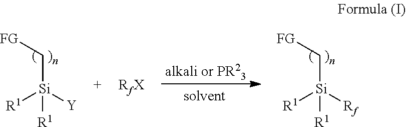
Wherein,
FG is halogen, OMs, OTs, NO₂, CF₃, CN, CO₂R, CONR₂, —CH═CR₂, —C≡CR, R is H, C₁₋₁₀ alkyl, C₁₋₁₅ aromatic ring, thiophene, furan, pyrrole, pyridine;
R_{f} is a C₁₋₁₀ alkyl group containing fluorine atoms;
R¹ is C₁₋₁₀ alkyl, aryl, the aryl is the electron donating group substituted benzene ring, the electron withdrawing group substituted benzene ring, naphthyl, thiophene, furan, pyrrole, pyridine, ester group; wherein, the electron donating group includes C₁₋₁₀ alkyl, C₁₋₁₀ alkoxy, the electron withdrawing group includes trifluoromethyl, ester group, nitro, cyano, halogen;
Y is halogen, OTf;
n=1-10;
X is H, halogen.

4. The method according to claim 3, wherein, FG is F, Cl, Br, I, OMs, OTs, NO₂, CF₃, CN, CO₂R, CONR₂, —CH═CR₂, —C≡CR, wherein, the R is H, C₁₋₁₀ alkyl, C₁₋₁₅ aromatic ring, thiophene, furan, pyrrole, pyridine; R_{f} is CF₃, CF₂H, CFH₂, C₂F₅, CF₂CF₂H, CF₂CF₂Cl, CF₂CF₂Br, CF₂CH₃, C₃F₇, CF₂CF₂CF₂H, CF₂CF₂CH₃, CF₂CH₂CH₃, C₄F₉, CF₂CF₂CF₂CF₂H, CF₂CF₂CF₂CH₃, CF₂CF₂CH₂CH₃, CF₂CH₂CH₂CH₃; R¹ is C₁₋₁₀ alkyl, electron donating group substituted benzene ring, electron withdrawing group substituted benzene ring, naphthyl, thiophene, furan, pyrrole, pyridine, ester group; wherein, the electron donating group includes methyl, methoxy, the electron withdrawing group includes trifluoromethyl, ester group, nitro, cyano, fluorine, chlorine, bromine, iodine; Y is Cl, Br, I, OTf; n=1-10; X is H, Br, I.

5. The method according to claim 3, wherein, the alkali is one or more of the following: lithium bis(trimethylsilyl) amide LiHMDS, potassium bis(trimethyl silyl) amide KHMDS, sodium bis(trimethylsilyl) amide NaHMDS, sodium amide NaNH₂, sodium hydride NaH; and/or, R² is C₁₋₁₀ alkyl group, C₁₋₁₀ alkoxy group, C₁₋₁₀ alkylamine group, aryl, and the aryl is electron donating group substituted benzene ring, electron withdrawing group substituted benzene ring, naphthyl, thiophene, furan, pyrrole, pyridine, ester group; wherein, the electron donating group includes C₁₋₁₀ alkyl group, C₁₋₁₀ alkoxy group, the electron withdrawing group includes trifluoromethyl, ester group, nitro, cyano, halogen.

6. The method according to claim 3, wherein, the reaction temperature is −78˜100° C.; and/or the reaction time is 2˜36 hours.

7. The method according to claim 3, wherein, the molar ratio of the fluoroalkyl source R_{f}X, the halosilane compound, the alkali or the tertiary phosphine compound PR²₃ is R_{f}X: halosilane compound: alkali or tertiary phosphine compound PR²₃=(1-20):(1-3):(1-3).

8. The method according to claim 3, wherein, the solvent is any one or more of the following: benzonitrile, phenylacetonitrile, acetonitrile, dichloromethane, toluene, tetrahydrofuran THF, diethyl ether, dimethylformamide DMF, dimethylacetamide, dimethyl sulfoxide DMSO, N-methylpyrrolidone NMP, hexamethylphosphoric triamide HMPA.

9. The functionalized fluoroalkyl silane compound synthesized by the method according to claim 3.

10. The application of the functionalized fluoroalkyl silane compound according to claim 1 in silylation reaction and functional group transfer reaction.

11. The functionalized fluoroalkyl silane compound synthesized by the method according to claim 4.

12. The functionalized fluoroalkyl silane compound synthesized by the method according to claim 5.

13. The functionalized fluoroalkyl silane compound synthesized by the method according to claim 6.

14. The functionalized fluoroalkyl silane compound synthesized by the method according to claim 7.

15. The functionalized fluoroalkyl silane compound synthesized by the method according to claim 8.

16. The application of the functionalized fluoroalkyl silane compound according to claim 2 in silylation reaction and functional group transfer reaction.

17. The application of the functionalized fluoroalkyl silane compound according to claim 9 in silylation reaction and functional group transfer reaction.
